(19) United States
(12) Patent Application Publication
(10) Pub. No.: US 2022/0354766 A1
(43) Pub. Date: Nov. 10, 2022

(54) FORMULATION FOR TOPICAL APPLICATION TO THE SKIN OR MUCOUS MEMBRANES

(71) Applicant: Sandrine SEBBAN, Levallois-Perret (FR)

(72) Inventor: Sandrine SEBBAN, Levallois-Perret (FR)

(21) Appl. No.: 17/763,518

(22) PCT Filed: Sep. 25, 2020
(86) PCT No.: PCT/EP2020/076913

§ 371 Date: Mar. 24, 2022

(30) Foreign Application Priority Data
Sep. 26, 2019 (FR) FR1910660

(51) Int. Cl. A61K 8/66; A61K 8/92; A61K 8/73; A61K 8/46; A61K 8/04; A61Q 19/00

(52) U.S. Cl. CPC A61K 8/66; A61K 8/92; A61K 8/73; A61K 8/463; A61K 8/04; A61Q 19/007

(57) ABSTRACT

The invention relates to a formulation for topical application to the skin or mucous membranes. The formulation has both a pulverulent composition with an enzymatic complex as active substance, and a liquid composition intended to be mixed with the pulverulent composition so as to obtain the formulation to be applied. The liquid composition comprises a solvent and in addition, an agent for dispersing the pulverulent composition in the liquid composition. The invention also relates to a system for packaging and dispensing the formulation.

### CROSS-REFERENCE TO RELATED APPLICATIONS

This application claims the benefit of International application number PCT/EP2020/076913, filed Sep. 25, 2020 and French patent application number 1910660, filed on Sep. 26, 2019, the entire contents of which are incorporated herein by reference.

### TECHNICAL FIELD

The invention relates to a formulation comprising an enzymatic complex for topical application to the skin or mucous membranes, in particular in the context of a medical and/or aesthetic intervention, as well as a system for packaging and dispensing such a formulation for topical application.

BACKGROUND

According to one embodiment, the enzymatic complex contains in particular hyaluronidase. The complex may comprise other enzymes, such as lipase, protease, bromelain and/or Q10 coenzyme.

Hyaluronic acid is a biological product widely present in human and animal tissues, in particular in the skin, where it fills the intercellular spaces and participates in particular in the hydration and cohesion of the tissues.

Moreover, hyaluronidase is an enzyme or a group of enzymes capable of degrading hyaluronic acid, thus increasing the permeability of tissues. In the natural state, hyaluronidases are found for example in the plasma membrane of spermatozoa, which enables them to penetrate an ovum by degrading the hyaluronic acid present in the protective layers of said ovum.

Because of this property, the use of hyaluronidase is frequent in medicine, especially in combination with other drugs to accelerate their dispersion in the organism.

Hyaluronidase is also widely used as an injection in the field of aesthetic medicine, in particular as a complement to injections of filler products based on hyaluronic acid, to correct effects considered as unsightly or in the case of side effects and/or complications resulting from said injections of hyaluronic acid.

Indeed, if the filler product is injected in too large amount, too heterogeneously and/or in a badly chosen area of the skin, the treated skin could have correction defects conferring thereon an unnatural appearance, in particular asymmetries and/or swelling, which is all the more detrimental if the injections were performed on the face.

Furthermore, such injections could cause side effects and/or medical complications which might be dangerous to health, such as oedemas, embolisms, etc.

Thus, to correct these side effects, it is possible to treat the skin with hyaluronidase, in particular by injection into said skin of a formulation containing it, as explained for example in the following articles:

“Hyaluronidase in the correction of hyaluronic acid-based fillers: a review and a recommendation for use” (RZANY, BECKER-WEGERICH, BACHMANN, ERDMANN, WOLLINA, Journal of Cosmetic Dermatology, December 2009);

“The Kinetics of Reversible Hyaluronic Acid Filler Injection Treated With Hyaluronisade” (JUHASZ, LEVIN, MARMUR, Dermatologic Surgery, Volume 43(6), pp. 841-847, American Society for Dermatology Surgery, June 2017);

“Hyaluronidase in cosmiatry: what should we know?” (ADA REGINA TRINDADE DE ALMEIDA, ANA FLAVIA NOGUEIRA SALIBA, Surgical and Cosmetic Dermatology, Volume 7(3), January 2015).

In particular, such a formulation may be prepared by mixing in a suitable solvent a pulverulent composition comprising hyaluronidase, said mixing being preferably performed extemporaneously to preserve the stability of the hyaluronidase enzyme(s) until use thereof.

To limit the risk of trauma associated with subcutaneous injections (hematomas, bruises, oedemas, etc.), it is also possible to consider administering hyaluronidase by topical application of a suitable formulation on the external layer of the skin or on the mucous membranes.

In particular, in the case of injections, the most frequently reported undesirable effects are moderate local reactions at the injection site: irritation, infection, bleeding or hematoma, softening of the skin, wilting. Serious allergic reactions including anaphylactic reactions have been reported. In addition, allergic reactions have included cases of periorbital oedema with the use of hyaluronidase associated with local anaesthetics in ophthalmology. Cases of exophthalmos (proptosis) have been reported. Several cases of facial angioedema have been observed in ophthalmic surgery with a favourable outcome. Oedemas have been reported associated to a subcutaneous injection.

Thus, the document WO-2009/037566 provides for the use of a particular hyaluronidase in a formulation in the form of a lotion or a cream for topical application, in particular for aesthetic purposes such as:

the prevention or reduction of wrinkles;

the prevention or attenuation of scars, in particular due to surgical incisions, burns or acne;

the stimulation or attenuation of capillary and/or hair growth, in combination with an appropriate active substance.

To correct defects and/or side effects due to injections of hyaluronic acid, it is necessary to administer the formulation containing hyaluronidase to very specific areas of the skin or mucous membranes to be treated, which in practice turns out to be difficult to achieve with a formulation for topical application.

In particular, the formulation should have a viscosity high enough so as not to flow after application thereof, but also remain fluid enough to facilitate application and penetration thereof.

To this end, it is possible to consider adapting the viscosity of the formulation by adding thereto a consistency agent.

Nonetheless, this solution does not give full satisfaction, to the extent that it could cause problems of dispersion of the hyaluronidase powder in the solvent, which is not desirable.

### SUMMARY OF THE INVENTION

The invention aims to improve the prior art by proposing in particular a formulation containing an enzymatic complex which has a satisfactory viscosity to enable topical application thereof in a very accurate manner to the skin or the mucous membranes, while being adapted to guarantee a homogeneous dispersion of said enzymatic complex in said formulation.

To this end, according to a first aspect, the invention provides a formulation for topical application to the skin or mucous membranes, said formulation comprising, on the one hand, a pulverulent composition comprising an enzymatic complex as active substance and, on the other hand, a liquid composition intended to be mixed with said pulverulent composition so as to obtain the formulation to be applied, said liquid composition comprising a solvent, the liquid composition and/or the pulverulent composition comprising, in addition, an agent for dispersing the pulverulent composition in said liquid composition.

According to a second aspect, the invention provides a system for packaging and dispensing such a formulation, said system comprising a body equipped with a device for carrying out said dispense for the topical application of said formulation.

A formulation for topical application to the skin or mucous membranes is described hereinbelow, in particular in the context of a medical and/or aesthetic intervention, as well as a system for packaging and dispensing such a formulation.

The formulation comprises, on the one hand, a pulverulent composition comprising an enzymatic complex as active substance and, on the other hand, a liquid composition intended to be mixed with said pulverulent composition so as to obtain the formulation to be applied. In particular, the formulation to be applied is in the form of a cream or a lotion.

According to one embodiment, the enzymatic complex contains in particular hyaluronidase. The formulation could then be applied to the skin or mucous membranes for aesthetic purposes, in particular as a complement to injections of hyaluronic acid-based filler products.

The enzymatic complex may also comprise, alone or in combination, other enzymes, such as:

lipase which is used in anti-cellulite products to facilitate the elimination of fatty deposits, used in enzymatic peelings to eliminate sebum;

protease which is used in enzymatic peelings to eliminate dead skins and promote cell renewal and therefore skin regeneration. It also promotes tissue permeability;

bromelain derived from pineapple, which has a proteolytic action similar to the mechanism of action of protease;

the Q10 coenzyme, a powerful antioxidant, restoring tissue DNA and increasing cell oxygenation.

The enzymatic complex may comprise between 90% and 100% by weight of a compound containing dosed hyaluronidase, for example in maltodextrin, at 0.01% on average, in particular more than 95% by weight of such a compound. According to one embodiment, the enzymatic complex also comprises less than 1% by weight of a second compound containing lipase and protease each dosed at 0.01% on average. This enzymatic complex may in particular comprise a weight ratio of these two compounds of 99.5:0.5, thus promoting the permeability of the tissues.

A formulation containing hyaluronidase could be used in particular for the following aesthetic treatments, without being limited thereto:

the prevention or the reduction of wrinkles;

the prevention or the attenuation of scars, in particular due to surgical incisions, burns or acne;

the stimulation or the attenuation of capillary and/or hair growth, in combination with an appropriate active substance;

the treatment of cellulite.

The formulation could also be used to correct side effects due to such injections of hyaluronic acid, in particular defects in the correction of the skin such as swelling and/or asymmetries, and/or to treat medical complications, such as oedemas, which might potentially be dangerous to health, such as embolisms, etc.

The formulation may comprise between 1% and 25%, and preferably between 3% and 16% by weight of enzymatic complex, depending on the medical and/or aesthetic treatment that we wish to perform. In particular, the results of the performed treatment are proportional to the used concentration degrees and to the used amount. Furthermore, the topical application should be done in an extremely accurate manner on the considered area because of the diffusion effect of the formulation.

As example, it is possible to provide for formulations comprising an enzymatic complex containing hyaluronidase in the following proportions:

to treat immediate post-injection oedema or mucous membranes in the lip area: about 4% by weight of enzymatic complex;

to treat an excess of an injection of hyaluronic acid or the cheekbone area: about 8% by weight of enzymatic complex;

to treat a serious medical complication such as an embolism, in complement to the hyaluronidase injection: about 15% by weight of enzymatic complex.

Advantageously, the enzymatic complex comprises hyaluronidase in liposomal form, which allows improving the preservation and the cutaneous penetration of said hyaluronidase.

Indeed, liposomes are spherical lipid vesicles which, once formed, are stable structures, and whose membrane consists of one or several bilayer(s) of phospholipids with a structure biomimetic to that of the superficial layers of the epidermis. Thus, liposomes have the ability to penetrate the skin particularly well.

The formulation comprises:

between 3% and 20% by weight of a pulverulent composition; and

between 80% and 97% by weight of a liquid composition.

With such amounts of pulverulent composition, the formulation may have a pasty consistency which, in addition to conferring thereon a relatively unpleasant appearance, may complicate not only the dispense thereof, for example by forming residues in the system used for said dispense, but also its topical application.

To overcome these drawbacks, the pulverulent composition comprises an anti-caking agent, in order to avoid the formation of lumps during mixing thereof with the liquid composition.

In particular, the formulation comprises between 1% and 10%, preferably between 2% and 5%, for example about 3% by weight of an anti-caking agent, in particular based on dictamen root extract (Maranta arundinacea), starch, in particular rice starch (Oryza sativa starch) and/or talc.

The liquid composition comprises a solvent, in particular demineralised water.

According to one embodiment, the formulation comprises between 40% and 90%, and preferably between 50% and 75% by weight of a solvent.

Moreover, the liquid composition and/or the pulverulent composition comprises an agent for dispersing the pulverulent composition in said liquid composition.

Thus, a formulation is obtained with a satisfactory viscosity to enable topical application thereof in a very accurate manner to the skin or the mucous membranes, while being suitable for guaranteeing a homogeneous dispersion of the enzymatic complex in said formulation.

Advantageously, the liquid composition comprises an additional active substance based on an essential oil or a mixture of essential oils, the dispersing agent being present in said liquid composition to improve the dispersion of said additional active substance in said liquid composition.

To this end, the dispersing agent may comprise:

between 50% and 70% by weight of glycerine;

between 20% and 30% by weight of a solvent, in particular a water-based one;

between 1% and 10% by weight of an emulsifying agent, in particular a soy lecithin-based one.

For example, such a dispersing agent is commercialised under the trademark Solubol®, and may also have, as the case may be:

pumpkin and coconut natural extracts;

between 0.01% and 0.1% by weight of ascorbic acid;

between 0.01% and 0.1% by weight of citric acid;

between 0.01% and 0.1% by weight of tocopherol;

between 0.01% and 0.1% by weight of officinal rosemary (Rosmarinus officinalis).

The formulation may also comprise a dispersing agent based on oleic glycerides, in particular selected from the range of products commercialised under the trademark Labrafil®, and more particularly Labrafil® M2125 CS.

The dispersing agent may comprise several products allowing synergistically improving the dispersion of the additional active substance in said liquid composition and/or of the pulverulent composition in said liquid composition in order to obtain a homogeneous mixture. Furthermore, at least one of the used products might confer an additional effect, such as improving skin hydration.

In particular, the formulation comprises between 2% and 40%, and preferably between 5% and 20% by weight of a dispersing agent.

The additional active substance comprises at least one essential oil which has at least one of the following properties: healing, antiseptic, anti-oedematous, haemostatic, anti-inflammatory and/or analgesic.

In particular, the additional active substance comprises at least one essential oil selected from Italian helichrysum essential oil (Helichrysum italicum or golden eternal flower essential oil), cistus ladaniferus essential oil (Cistus ladaniferus), myrrh essential oil (Commiphora myrrha), lavender essential oil (Lavandula angustifolia), frankincense essential oil (Boswellia carterii or Boswellia serrata), gaultheria essential oil (Gaultheria procumbens), tea tree essential oil (Melaleuca alternifolia), rose geranium essential oil (Pelargonium graveolens), or a mixture of at least two of these components.

Advantageously, the formulation comprises:

between 0.1% and 10%, and preferably between 0.5% and 5% by weight of Italian helichrysum essential oil, for its anti-oedematous, healing and antiseptic properties;

between 0.1% and 10%, and preferably between 0.25% and 2.5% by weight of cistus ladaniferus essential oil, for its haemostatic, healing and antiseptic properties;

between 0.1% and 10%, and preferably between 0.25% and 2.5% by weight of myrrh essential oil, for its analgesic and healing properties;

between 0.1% and 10%, and preferably between 0.25% and 2.5% by weight of lavender essential oil, for its restorative (fight against skin irritation), skin regenerating, purifying properties.

In particular, the content of essential oil(s) in the formulation is suitable to confer thereon the desired active properties, but should not be too high so as not to incommode with excessively strong odours, in particular in the event of application on the face.

The additional active substance comprises a complex of essential oils which, by its amphiphilic properties, greatly improves the cutaneous penetration of the formulation. In particular, the formulation may comprise between 1% and 5% by weight of a complex of essential oils.

Depending on the intended use for the formulation, the liquid composition may also comprise at least one other additional active substance which has at least one of the following properties: moisturising, soothing, anti-inflammatory, anti-oedematous, healing, restorative, antioxidant, anti-cellulite and/or anti-wrinkle.

This supplemental additional active substance may be based on aloe vera, polysaccharide and/or glycerine.

Advantageously, the formulation comprises:

between 0.01% and 5% by weight of a moisturising and/or soothing agent based on polysaccharide, in particular Fucogel®, which also has restorative properties and imparts a fresh effect when applied topically;

between 0.01% and 20% by weight of a moisturising and/or soothing agent based on aloe vera, in particular Aloe Vera AG014®, which also has anti-inflammatory and healing properties.

The formulation may also comprise between 1% and 50%, and in particular between 8% and 10% by weight of a moisturising and/or soothing agent based on glycerine. In particular, this agent may be based on vegetable glycerine.

The formulation also comprises:

between 0.1 and 5% by weight of an antimicrobial agent, in particular from a plant origin, for example based on sodium levulinate, sodium anisate, chlorhexidine and/or glyceryl caprylate;

between 0.1% and 2% by weight of a gelling agent, in particular based on a vegetable gum, and preferably based on xanthan gum.

### Formulation Example

In connection with the attenuation of the imperfections related to the presence of hyaluronic acid resulting in undesirable visible signs, the following formulation has been made.

| | | | Weight |
|---|---|---|---|
| | | | per- |
| | Ingredients | Main properties | centage |
| Pulverulent | Enzymatic complex: | Depolymerisation of | 15.00% |
| composition | 99.5% of a compound | the injected | |
| | containing hyaluronidase | hyaluronic acid + | |
| | dosed at 0.01% in | Promote the | |
| | average and 0.5% of a | permeability of the | |
| | compound containing | tissues | |
| | lipase and protease each | | |
| | dosed at 0.01% in | | |
| | average | | |
| | Vegetable anti-caking | Dispersion of the | 3.00% |
| | agent: Oryza sativa | enzymatic complex, | |
| | starch | improved rheology | |
| Liquid | Extract of natural | Freshness feeling | 0.44% |
| composition | polysaccharides | Soothing, | |
| | (Biosaccharide gum-1) | moisturising, anti- | |
| | | ageing | |
| | Vegetable glycerine | Humectant, | 8.81% |
| | | moisturising | |
| | Aloe Vera concentrate | Soothing, healing, | 4.40% |
| | | moisturising | |
| | Complex of essential oils | Anti-oedematous, | 2.21% |
| | of golden eternal flower | Healing, Antiseptic + | |
| | (40%), lavender (20%), | amphiphilic | |
| | cistus (20%) and myrrh | properties, high | |
| | (20%) | cutaneous | |
| | | penetration | |
| | Vegetable dispersant: | Dispersion of the | 7.05% |
| | Solubol | complex of essential | |
| | | oils | |
| | Co-dispersant: | Facilitates the | 4.40% |
| | Pentylene glycol | dispersion of the | |
| | | essential oils and the | |
| | | obtainment of a | |
| | | homogeneous | |
| | | mixture. Additional | |
| | | effect: improves the | |
| | | hydration of the skin. | |
| | Vegetable antimicrobial | Preserve the | 3.09% |
| | agents: sodium | formulation from the | |
| | levulinate, sodium | microbial proliferation | |
| | anisate, glyceryl | (preservation) | |
| | caprylate | | |
| | Vegetable gelling agent: | Texturing agent | 0.44% |
| | xanthan gum | | |
| | Vegetable pH regulator: | Allows adjusting the | 0.06% |
| | lactic acid | pH of the formula | |
| | Water | Solvent | 51.08% |

### Selected Enzymes

The first selected enzyme is hyaluronidase. The use of this enzyme allows increasing the permeability of the skin and of the connective tissue. Already naturally present in the skin, this enzyme catalyses the hydrolysis of the glycosidic bond of hyaluronic acid. It allows depolymerising hyaluronic acid, and therefore reducing cross-linking thereof and thus the volume of the considered cutaneous surface. Hyaluronic acid will then be more easily degraded and eliminated by natural routes, through the connective tissue. This enzyme has the direct consequence of reducing the volume related to hyaluronic acid.

The second enzyme is lipase, already naturally present in the skin, it targets lipids and thus will make the surface of the skin (horny layer), which is essentially made up of fatty substances, more permeable. Thus, it will facilitate the action of hyaluronidase.

The third enzyme is protease, already naturally present in the skin, it has an action on proteins and will also improve the permeability of tissues such as the skin. Thus, it will facilitate the action of hyaluronidase.

### Selected Essential Oils

In order to complete but also to potentiate the action of the enzymatic complex, a complex of essential oils has been selected.

In general, essential oils have amphiphilic properties which confer on them the ability to facilitate the permeability of the skin and thus to promote the action of the active ingredients of a formula.

In addition, the selected essential oils have additional properties that complete the field of action of the enzymatic complex:

golden eternal flower essential oil: revitalising (reduces redness, swelling), skin regenerating and purifying;

myrrh essential oil: soothing (calms skin discomfort feelings), skin restorative, purifying;

officinal lavender essential oil: restorative (fights skin irritation), skin regenerating, purifying;

cistus essential oil: astringing (firms the skin), skin regenerating, purifying.

Each has different and complementary main actions that reinforce the effects of the formulation.

Also, all of them have a purifying action to cleanse the skin and a regenerating action to improve the elasticity and firmness of the skin in order to reinforce the skin perfecting effect related to the enzymatic complex.

### Completed Tests

Tests on corpses have been carried out to test the effectiveness of the mixed formulation according to the example hereinabove.

### Operating Procedure

Biopsies have been carried out by a dissector anatomist. Afterwards, the samples have been sent to an anatomical pathology laboratory where the samples have been fixed, sectioned and stained so that the results could be observed under the microscope.

Several histological sections have been made. Staining allows visualising the skin layers present in the sample (epidermis, dermis, hypodermis, muscle tissue). Alcian blue stain is used to stain glycoproteins, including hyaluronic acid.

### Experiment A

A superficial injection of 1 ml of hyaluronic acid (Stylage M) has been carried out in the right malar area with a 27 G 13 mm needle. This same injection is also carried out in the left malar area to have an equivalence in terms of injected product and treated area. Afterwards, a pipette (about 0.54 g) of formulation according to the example is applied only to the injected left malar area so as to be able to observe its effectiveness in comparison with the reference right malar area.

We observe, with the naked eye, a lower presence of hyaluronic acid on the slides of the histological sections of the left malar area in comparison with those of the right malar area. Visually, the bolus created by the injection of hyaluronic acid has disappeared after application of the formulation. In addition, on the corpse, the disappearance of the created swelling is noticed.

### Experiment B

On the lower lip, three injections of 0.5 ml of hyaluronic acid (Juvederm Ultra 3) have been performed to the right, at the centre and to the left. 0.1 ml of injectable hyaluronidase (Desinfiltral), diluted beforehand in 3 ml of physiological saline, have been injected into the left side. On the right side is applied 1 pipette (about 0.54 g) of formulation according to the example.

At the centre, there is a presence of hyaluronic acid in the muscle tissue, and in a smaller amount in the superficial dermis, the middle dermis and the deep dermis. To the left and to the right, the presence of hyaluronic acid in the different layers of the dermis has completely disappeared. Only hyaluronic acid remains in the muscle tissue.

### Experiment C

Two injections of 0.5 ml of hyaluronic acid (Juvederm Ultra 3) have been performed in the nasolabial folds, to the left and to the right. 0.1 ml of Desinfiltral, diluted beforehand in 3 ml of physiological saline, have been injected into the left side. On the right side is applied 1 pipette (about 0.54 g) of formulation according to the example.

To the left, there is a small amount of hyaluronic acid in the hypodermis, whereas to the right, there is no more hyaluronic acid after application of the formulation according to the invention. Thus, the effects of the formulation according to the invention are equivalent or still better than those conferred by the use of Desinfiltral.

Another study on a patient has been carried out in collaboration with Canfield. The use of the VECTRA H2 apparatus with high-precision optical properties allows for high-resolution 3D images (colour resolution: 18% Mega pixels, geometric resolution: 0.95 mm) in order to visualise the topography of the skin, in particular its relief.

In the context of a treatment, 3D images are captured before and after. Once the images are available, the difference in volume between two images can be measured by visualising the degree of contour change thanks to a colour relief map.

The patient in her sixties had an overcorrection at the lower eyelid.

### Operating Procedure

Once the pulverulent composition has been mixed with the liquid composition, 4 drops corresponding to about 0.24 g of formulation have been deposited accurately on the lower eyelid. Between each deposited drop, the formulation is applied locally using a brush, by performing circular movements. Afterwards, a massage is performed for about 3 minutes with gloved fingers, then with the SoftFil® Skin Massager.

Front, profile and ¾ photos have been captured with the VECTRA H2 apparatus before and after completion of the operating procedure hereinabove.

The volume loss is visibly noticeable. This volume loss has been quantified using the VECTRA H2 apparatus and the results have shown:

a loss of 1.27 mm in surface projection;

a loss of 0.27 cm³ in volume.

### BRIEF DESCRIPTION OF THE DRAWINGS

Other particularities and advantages of the invention will appear in the following description, made with reference to the appended figures, wherein:

FIGS. 1a and 1b schematically represent in longitudinal section a system according to a first embodiment of the invention, respectively before (FIG. 1a) and after (FIG. 1b) the displacement of the breaking device on the use stroke;

FIG. 1c represents in perspective top view the breaking device of FIGS. 1a and 1b;

FIGS. 2a and 2b schematically represent in longitudinal section a system according to a second embodiment of the invention, respectively before (FIG. 2a) and after (FIG. 2b) the displacement of the breaking device on the use stroke;

FIGS. 3a, 3b and 3c schematically represent examples of applicators that could be associated with the dispensing device of the systems of the preceding figures for performing the topical application of the formulation after mixing of the compositions.

### DETAILED DESCRIPTION

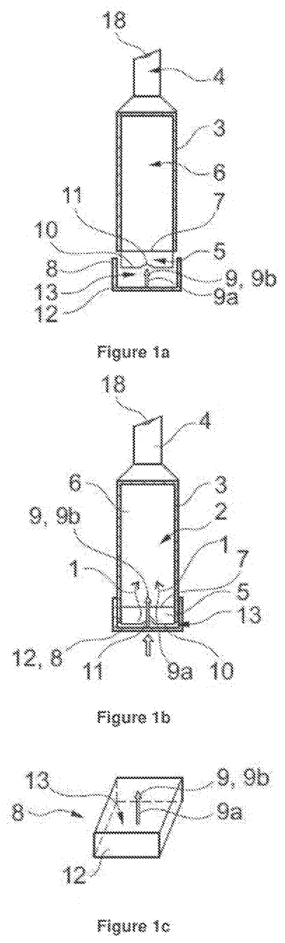

Referring to FIGS. 1 to 3, a system for packaging and dispensing the previously-described formulation is now described. In particular, the system may be packaged as a whole or separately, in a blister, whether sterile or not, before use thereof.

In general, the system comprises a body 3 equipped with a device 4 for carrying out the dispense of the formulation for topical application thereof.

In a particular application, the body may be in the form of a one-use patch which comprises a matrix in which the formulation is impregnated, said matrix being intended to be applied on an area to be treated to simultaneously ensure the dispense and the topical application of said formulation.

According to another particularly advantageous embodiment, represented in the figures, the body 3 has a first 5 and a second 6 separate reservoirs for respectively packaging one amongst the pulverulent composition and the liquid composition, said system further comprising means for obtaining said formulation by extemporaneous mixing of said compositions in said body. Indeed, an extemporaneous mixing of the compositions allows preserving the stability of the enzyme(s) of the complex, in particular hyaluronidase, until use thereof, and thus benefiting from a formulation with an optimum enzyme stability at the time when the desired medical and/or aesthetic treatment should be performed.

For this purpose, the reservoirs 5, 6 are separated by a frangible wall 7, the system comprising a device 8 for breaking up at least partially said frangible wall in order to set said reservoirs in communication to obtain said formulation.

In the figures, the body 3 comprises a first upstream reservoir 5 in which the pulverulent composition 1 is packaged and a second downstream reservoir 6 in which the liquid composition 2 is packaged. Alternatively, the liquid composition could be packaged in the upstream reservoir 5 and the pulverulent composition in the downstream reservoir 6.

The breaking device 8 comprises a piercing means 9 which is mounted on the body 3 in a departed position of the frangible wall 7, said breaking device being movable over a use stroke on which the piercing means 9 crosses the first reservoir 5 to come into the second reservoir 6 throughout the frangible wall 7.

In particular, the frangible wall 7 and/or the piercing means 9 are arranged so as to obtain a tearing of said frangible wall by said piercing means. In particular, the piercing means 9, and in particular its tip 9b, may be arranged so that its passage wrecks the frangible wall in order to facilitate the release of the first composition 1 into the second reservoir 6.

The frangible wall 7 may be made of an easily tearable material, in particular a waterproof sheet, based on plastic and/or aluminium, for example by having a tension facilitating tearing thereof by the piercing means 9.

Advantageously, the piercing means 9 comprises a rod 9a whose distal end is surmounted by a tip 9b, said tip being sharp enough to ensure piercing of the frangible wall 7 without the need for a significant effort from the user.

Referring to FIG. 1c, the tip 9b has a base surmounted by a head, said base having an external dimension which is larger than the external dimension of the rod 9a.

Thus, after passage of the point 9b, the frangible wall 7 has a tear with size large enough to enable the rod 9a to pass easily throughout it, and thus the piercing means 9 to continue its use stroke throughout the first reservoir 5.

Advantageously, the tip 9b has a conical shape, which enables a progressive tearing of the frangible wall 7 around said tip at the beginning of the use stroke.

Referring to the figures, the piercing means 9 in the departed position is disposed opposite an outer wall 10 of the first reservoir 5, said outer wall being arranged so as to enable the passage of said piercing means throughout it at the beginning of the use stroke.

The outer wall 10 could be pierceable by the piercing means, in particular by being made of a suitable material and/or by having a reduced thickness at least opposite the piercing means 9. The outer wall 10 may also have an orifice through which the piercing means 9 passes on its use stroke, said orifice possibly being covered with a lid before said passage to prevent leakages of the composition 1 out of the first reservoir 5.

In particular, as represented in FIGS. 1a and 1b, the outer wall 10 may have a concave central area 11 specifically arranged so as to enable the passage of the piercing means 9.

In FIGS. 1a, 1b and 1c, the system is in the form of a one-use vial, in particular arranged so as to contain a dose of formulation of less than 5 millilitres.

In particular, the body 3 of the vial may have a circular or square section, as well as the following dimensions:

a longitudinal dimension in the range of 5+/−1 centimetres;

a transverse dimension in the range of 1.5 cm.

In this embodiment, the breaking device 8 comprises a support 12 on which the piercing means 9 is associated, said support being arranged to be able to slide on the body 3 in order to move said piercing means over its use stroke.

The support 12 comprises a housing 13 at the centre of which the piercing means 9 is disposed, said housing having an inner geometry which is complementary to that of the bottom of the body 3 so as to be embedded under said body at the end of the use stroke (FIG. 1b).

In particular, given the dimensions of the body 3, the support 12 has a height in the range of 1 cm.

The support 12 could be slidably associated on the body 3, which allows improving the ergonomics of use of the system. In this case, the breaking device 8 may be equipped with a safety means to prevent any inadvertent movement of the piercing means 9 before using the system, the user deactivating said safety means when he wishes to mix the compositions 1, 2.

Alternatively, the body 3 could be mounted on a separate support for moving the piercing means 9 over its use stroke. Thus, the user should assemble the body 3 to the breaking device 8 when he wishes to perform mixing of the compositions 1, 2.

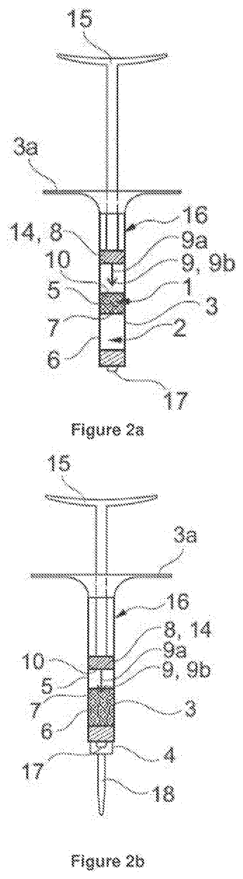

In FIGS. 2a and 2b, the system is in the form of a syringe. In particular, the body 3 of the syringe may have the following geometric arrangements:

a longitudinal dimension in the range of 6+/−0.5 centimetres;

a diameter in the range of 1+/−0.1 centimetres; and

a collar 3a for digital holding with a transverse dimension of about 4.5+/−0.5 centimetres.

In this embodiment, the breaking device 8 comprises a plunger 14 at the centre of which the piercing means 9 is associated, said plunger being arranged so as to be able to slide in the body 3 in order to move said piercing means over its use stroke.

To enable the movement of the piercing means 9, the plunger 14 is mounted on a syringe pusher 15, whose transverse dimension is about 5.5+/−0.5 centimetres.

Moreover, the body 3 has a third reservoir 16, in particular formed upstream of the first reservoir 5, in which the piercing means 9 is disposed in the departed position.

As represented in FIGS. 2a and 2b, the second reservoir 6 is in communication with an orifice 17 for dispensing the formulation, a suitable device 4 being mounted on the system to be able to dispense an amount of formulation coming from said orifice. The piercing means 9 could fit into the dispensing orifice 17 at the end of its stroke in order to promote mixing and dispensing of all of the formulation.

Advantageously, the dispensing device 4 is equipped with an applicator 18 to enable the topical application of the formulation directly to a portion of the body to be treated, in particular in the context of a medical and/or aesthetic intervention.

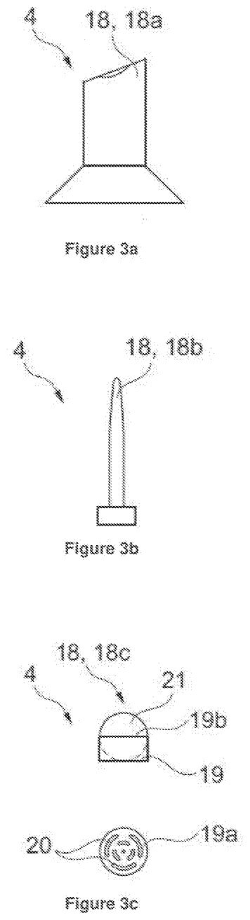

Referring to FIG. 3, this applicator 18 could in particular be selected from the group comprising a flat applicator 18a (FIGS. 1a, 1b, 3(a)), a drop-by-drop pipette 18b (FIGS. 2a, 2b, 3(b)) or a roll-on 18c.

In particular, as represented in FIG. 3(c), a roll-on type applicator 18c comprises a base 19 which is mounted on the dispensing orifice 17, said base comprising:

a lower wall 19a provided with orifices 20 through which an amount of formulation to be applied could pass;

an upper opening 19b in which a ball 21 is rotatably mounted to enable the application by means of said ball of an amount of formulation on an area to be treated.

Alternatively, the dispensing device 4 could be arranged so as to dispense a given amount of formulation in a one-use patch, the topical application of said formulation then being carried out by disposing said patch on the area to be treated.

For this purpose, the dispensing device 4 may for example be equipped with a drop-by-drop pipette 18b as described before.

What is claimed is:

1. A formulation for topical application to the skin or mucous membranes, the formulation comprising, a pulverulent composition comprising an enzymatic complex as a first active substance and, a liquid composition comprising a solvent, wherein the liquid composition or the pulverulent composition comprises a dispersing agent for dispersing the pulverulent composition in the liquid composition.

2. The formulation according to claim 1, wherein the liquid composition comprises a second active substance based on an essential oil or a mixture of essential oils, the dispersing agent being arranged to improve the dispersion of the second active substance in the liquid composition.

3. The formulation according to claim 2, wherein the second active substance comprises at least one essential oil which has at least one of the following properties: healing, antiseptic, anti-oedematous, haemostatic, anti-inflammatory or analgesic.

4. The formulation according to claim 2, comprising between 1% and 5% by weight of a complex of essential oils.

5. The formulation according to claim 1, comprising between 2% and 40% by weight of the dispersing agent.

6. The formulation according to claim 1, wherein the liquid composition comprises a second active substance which has at least one of the following properties: moisturising, soothing, anti-inflammatory, anti-oedematous, healing, restorative, antioxidant, anti-cellulite and/or anti-wrinkle, the active substance being in particular based on aloe vera, polysaccharide and/or glycerine.

7. The formulation according to claim 1, comprising between 0.1 and 5% by weight of an antimicrobial agent, based on sodium levulinate, sodium anisate, chlorhexidine or glyceryl caprylate.

8. The formulation according to claim 1, comprising between 0.1% and 2% by weight of a gelling agent based on vegetable gum.

9. The formulation according to claim 1, wherein the enzymatic complex contains hyaluronidase.

10. The formulation according to claim 9, wherein the enzymatic complex comprises hyaluronidase in liposomal form.

11. The formulation according to claim 1, comprising between 1% and 25% by weight of the enzymatic complex.

12. The formulation according to claim 1, wherein the enzymatic complex comprises at least one enzyme promoting tissue permeability.

13. The formulation according to claim 1, wherein the pulverulent composition comprises an anti-caking agent.

14. The formulation according to claim 1, comprising between 40% and 90% by weight of a solvent.

15. The formulation according to claim 1, wherein it comprises:
between 3% and 20% by weight of the pulverulent composition;
between 80% and 97% by weight of the 1 liquid composition.

16. A system for packaging and dispensing a formulation according to claim 1, the system comprising a body configured with a dispensing device topical application of the formulation.

17. The system according to claim 16, comprising a one-use patch, the dispensing device being configured to dispense the formulation onto the one-use patch for the topical application of the formulation by the patch.

18. The system according to claim 16, wherein the dispensing device is equipped with an applicator to enable the topical application of the formulation.

19. The system according to claim 17, wherein the body has a first and a second separate reservoirs, the first reservoir for packaging the pulverulent composition and the second reservoir for packaging the liquid composition, the system further comprising a wall and breaking device for obtaining the formulation by extemporaneous mixing of the compositions in the body.
